(19) United States
(12) Patent Application Publication
(10) Pub. No.: US 2011/0027347 A1
(43) Pub. Date: Feb. 3, 2011

(54) POLYMERSOMES AND METHODS OF MAKING AND USING THEREOF

(76) Inventors: You Han Bae, Salt Lake City, UT (US); Halqing Yin, San Lake City, UT (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 12/510,541

(22) Filed: Jul. 28, 2009

(51) Int. Cl. A61K 9/127; C07C 233/01; C07K 7/06; C07K 7/08; C07K 14/00

(52) U.S. Cl. 424/450; 564/192; 530/330; 530/329; 530/328; 530/327; 530/326; 530/324; 530/402

(57) ABSTRACT

Described herein is the synthesis and pharmacology of polymersomes containing one or more bioactive agents. The polymersome is generally derived from a polymer having the formula XY₂, wherein X includes a hydrophilic group and Y includes a hydrophobic group. Also described herein are methods for making and using the polymersomes as drug delivery devices.

### BACKGROUND

Liposomes prepared from natural or synthetic lipids have been extensively studied as drug carriers in cancer chemotherapy and other applications. One drawback to liposomes is that they suffer from a fast blood clearance by the reticuloendothelial system. To increase the circulating half-life, a surface modification approach is required through coupling water-soluble polymers such as poly(ethylene glycol) (PEG) to a small fraction of the lipids. This is still complicated because the low molecular weight of the lipids often leads to compromised membrane stability and quick release of encapsulated cargos. Thus, other delivery systems that do not possess these disadvantages would be desirable as drug delivery devices.

### SUMMARY

Described herein is the synthesis and pharmacology of polymersomes containing one or more bioactive agents. The polymersome is generally derived from a polymer having the formula XY₂, wherein X includes a hydrophilic group and Y includes a hydrophobic group. Also described herein are methods for making and using the polymersomes as drug delivery devices. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the structure of a polymersome and generic structure of the XY₂ polymer used to make the polymersome.

FIG. 2 shows an exemplary synthetic procedure for producing polymersomes described herein.

FIG. 3 shows an exemplary synthetic procedure for producing polymersomes described herein having a targeting group attached to the hydrophilic group PEG and pH-sensitive groups attached to the hydrophobic groups.

FIG. 4 shows representative plots of (a) pyrene excitation spectra and (b) variations of I₃₃₆/I₃₃₃ as a function of the copolymer concentration for PEG₄₅-b-(PLLA_{12.5})₂.

FIG. 5 shows (a) differential interference contrast (DIC) and (b) fluorescence micrographs of the polymersomes of PEG₄₅-b-(PLLA_{32.5})₂ observed by laser scanning confocal microscopy (LSCM). The inset of (b) shows the magnified image of a Nile-red/FITC-dextran loaded polymersome as indicated by the number “1”.

FIG. 6 shows fluorescence micrographs of the polymersomes for (a) PEG₄₅-b-(PLLA_{60.5})₂ and (b) PEG₄₅-b (PLLA_{12.5})₂ by LSCM.

FIG. 7 shows micrographs of microstructures for (a) PEG₄₅-b-PLLA₂₃ and (b) PEG₁₁₇-b-PLLA₃₄ observed by TEM.

FIG. 8 shows (a) the distribution of apparent hydrodynamic radius (R_{h,app}) at 90°; (b) dependence of apparent diffusion coefficient (Dₐₚₚ) on the scattering wave vector (q) measured by DLS; (c) q dependence of excess scattered intensity Iₑₓ(θ) obtained from SLS for the nano-sized polymersomes of PEG₄₅-b-(PLLA_{32.5})₂. (Solid lines indicate the best linear fit).

FIG. 9 shows micrographs of nano-sized polymersomes of PEG₄₅-b-(PLLA_{32.5})₂ by TEM (a) and cryo-TEM (b) respectively.

FIG. 10 shows (a) the distribution of apparent hydrodynamic radius (R_{h,app}) at 90° by DLS; (b) TEM micrographs of nano-sized polymersomes of PEG₁₁₇-b-(PLLA_{17.5})₂.

FIG. 11 shows (a) the distribution of apparent hydrodynamic radius (R_{h,app}) at 90° by DLS; (b) TEM Micrographs of nano-sized polymersomes of PEG₄₅-b-(PLLA_{32.5})₂ fabricated by nano-precipitation method.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or can not be substituted and that the description includes both unsubstituted lower alkyl and lower alkyl where there is substitution.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “aromatic group” as used herein is any group containing an aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “amino group” is defined herein as a primary, secondary, or tertiary amino group. In the alternative, the nitrogen containing substituent can be a quaternary ammonium group. The nitrogen containing substituent can be an aromatic or cycloaliphatic group, where the nitrogen atom is either part of the ring or directly or indirectly attached by one or more atoms (i.e., pendant) to the ring. The nitrogen containing substituent can be an alkylamino group having the formula —R—NH₂, where R is a branched or straight alkyl group, and the amino group can be substituted or unsubstituted.

The phrase “nitrogen containing group” is defined herein as any amino group. The nitrogen containing substituent can be a primary, secondary, or tertiary amino group. In the alternative, the nitrogen containing substituent can be a quaternary ammonium group. The nitrogen containing substituent can be an aromatic or cycloaliphatic group, where the nitrogen atom is either part of the ring or directly or indirectly attached (i.e., pendant) to the ring. The nitrogen containing substituent can be an alkylamino group having the formula —R—NH₂, where R is a branched or straight alkyl group, and the amino group can be substituted or unsubstituted.

Variables such as A, AA¹, AA², R¹—R¹⁸, T, X, Y, Z, a, b, and m used throughout the application are the same variables as previously defined unless stated to the contrary.

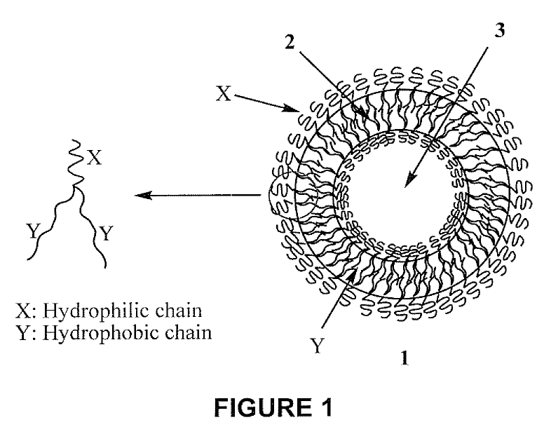

Described herein is the synthesis and pharmacology of polymersomes containing one or more bioactive agents. In one aspect, the polymersome includes one or more bioactive agents, wherein the polymersome is derived from a polymer having the formula XY₂, wherein X includes a hydrophilic group and Y includes a hydrophobic group. An exemplary structure of a polymersome 1 is provided in FIG. 1. Polymersomes are structurally different from other nanostructures such as nanospheres and micelles. In the case of nanospheres and micelles (e.g., spherical or worm-like), the core is not particularly defined or hollow. Polymersomes have a unique cell mimetic structure in which the polymers used to make the polymersome self-assemble into an enclosed bilayer 2 with an inner aqueous inner phase 3 (see FIG. 1). The term polymersome is also referred to as “vesicles.” The polymersome is produced from a polymer having the formula XY₂, wherein X includes a hydrophilic group and Y includes a hydrophobic group (FIG. 1). A detailed discussion regarding the polymers and methods for making the polymersomes is provided below.

The structure of polymer having the formula XY₂ is depicted below, where the polymer has a “Y” structure.

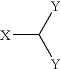

X and each Y can be bonded to one another by a variety of different groups that will be discussed below.

In the case of X, a variety of hydrophilic polymers can be used. In one aspect, the hydrophilic polymer includes, but is not limited to, poly(alkylene glycols), which can also be referred to as a poly(alkylene oxide) if the polymer was prepared from an oxide instead of a glycol. Other materials include a Pluronic™ F68 (BASF Corporation), a copolymer of polyoxyethylene and polyoxypropylene, which is approved by the U.S. Food and Drug Administration (FDA). As used herein, the term poly(alkylene glycol) refers to a polymer of the formula HO-[(alkyl)O]_{y}—OH, wherein “alkyl” refers to a C₁ to C₄ straight or branched chain alkyl moiety, including but not limited to methyl, ethyl, propyl, isopropyl, butyl, and isobutyl. In one aspect, y can be an integer greater than 4, and typically between 8 and 500, and more preferably between 40 and 500. Specific examples of poly(alkylene glycols) useful as a hydrophilic group include, but are not limited to, poly(ethylene glycol), polypropylene 1,2-glycol, poly(propylene oxide) and polypropylene 1,3-glycol. In one aspect, the hydrophilic group is PEG having a molecular weight of between approximately 500 to 20,000, 500 to 15,000, 500 to 10,000, 1,000 to 10,000, or 2,000 to 10,000.

Hydrophilic polymers other than a poly(alkylene glycol) that can be used include polypyrrolidone, poly(amino acids), including short non-toxic and non-immunogenic proteins and peptides such as human albumin, fibrin, gelatin and fragments thereof, dextrans, and poly(vinyl alcohol).

Turning to the hydrophobic polymer, in certain aspects it is bioerodible and biocompatible. Examples of hydrophobic polymers useful herein include, but are not limited to, copolymers of lactic acid and glycolic acid, as well as other polymers such as polyanhydrides, polyphosphazenes, polymers of α-hydroxy carboxylic acids, polyhydroxybutyric acid, polyorthoesters, polycaprolactone, polyphosphates, or copolymers prepared from the monomers of these polymers. Depending upon the application of the polymersomes, the hydrophobic groups present in the polymer can be the same or different polymer.

In one aspect, hydrophobic group includes, independently, polylactic acid or poly(lactic-co-glycolic) acid having a molecular weight from to 500 to 10,000, 500 to 9,000, 500 to 8,000, 500 to 7,000, 500 to 6,000, 500 to 5,000, 750 to 4,000, or 1,000 to 4,000.

In one aspect, the hydrophilic group X and hydrophobic groups Y can be attached via a trifunctional linker. The trifunctional linker is any compound that possesses three functional groups capable of forming a covalent bond with the hydrophilic and hydrophobic groups. Examples of such functional groups include, but are not limited to, an amino group, a hydroxyl group, a carboxylic group (e.g., ester or acid), a carbonyl group, a carbamate group, a ureyl group, a thiol group or an amide group. For example, when the trifunctional linker possesses an amino group, the amino group can react with a carboxylic acid group present on the hydrophilic or hydrophobic polymer to produce an amide bond, where the hydrophilic or hydrophobic polymer is covalently attached to the trifunctional linker. An example of a trifunctional linker useful herein is serinol. Other examples are discussed in detail below.

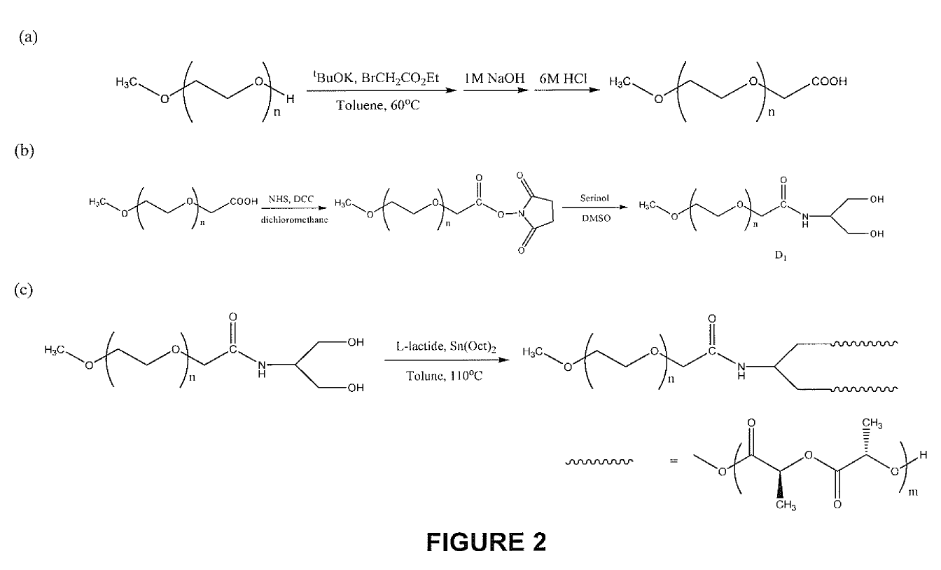

In one aspect, the polymer used to produce the polymersome has the formula I

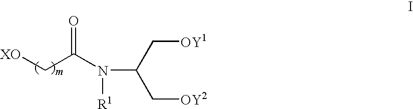

wherein X includes a hydrophilic group;
Y¹ and Y² include hydrophobic groups;
m is from 1 to 5; and
R¹ is hydrogen, an alkyl group, or an aryl group.
   In one aspect, referring to formula I, the hydrophilic group is polyethylene glycol, Y¹ and Y² are, independently, polylactic acid, polyglycolic acid, poly(lactic acid-co-glycolic acid), poly(ε-caprolactone); m is one, and R¹ is hydrogen. FIG. 2 and the Examples provide an exemplary procedure for making polymers having the formula I.

In certain aspects, one or more targeting groups can be bonded to the hydrophilic group. As used herein, “targeting group” includes any chemical moiety capable of binding to, or otherwise exhibiting an affinity for, a particular type of tissue or component thereof. The addition of a targeting group to the hydrophilic group can direct the resulting polymersome to particular sites within the body for targeted release of the bioactive agent present in the polymersome. For example, certain moieties are known to exhibit an affinity for hydroxyapatite surfaces (i.e. calcium phosphate), such as bone. Exemplary hydroxyapatite-targeting groups include tetracycline, calcein, bisphosphonates, such as 4-amino-1-hydroxybutane-1,1-diphosphonic acid, ditetrabutylammonium salt (AHBDP) or derivatives thereof, polyaspartic acid, polyglutamic acid, and aminophosphosugars. Additional targeting groups include proteins, antibodies, antibody fragments, peptides, carbohydrates, lipids, oligonucleotides, DNA, RNA, or small molecules having a molecular weight less than 2,000 Daltons.

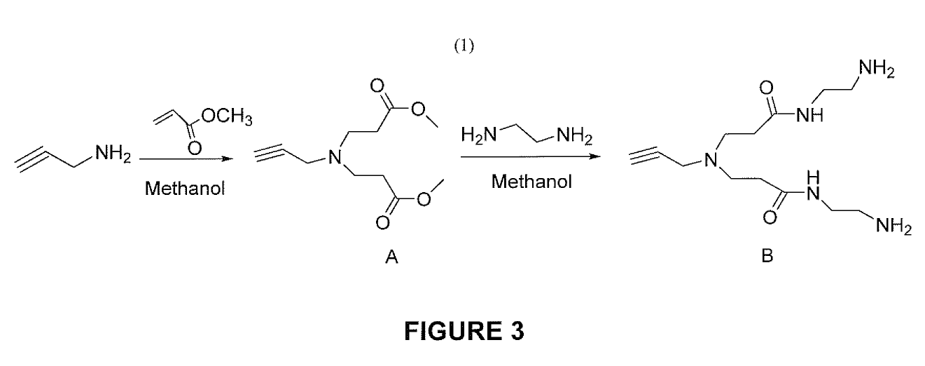

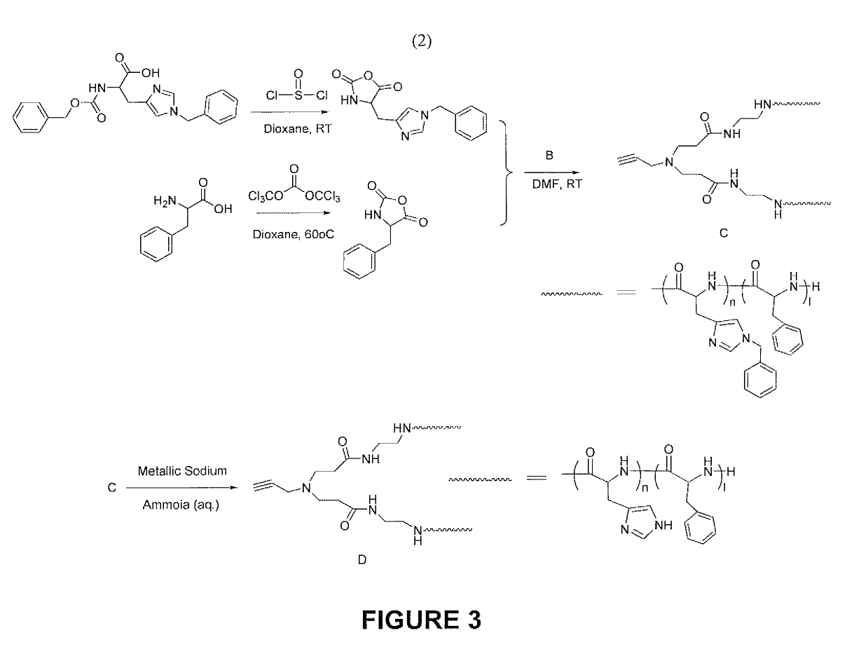

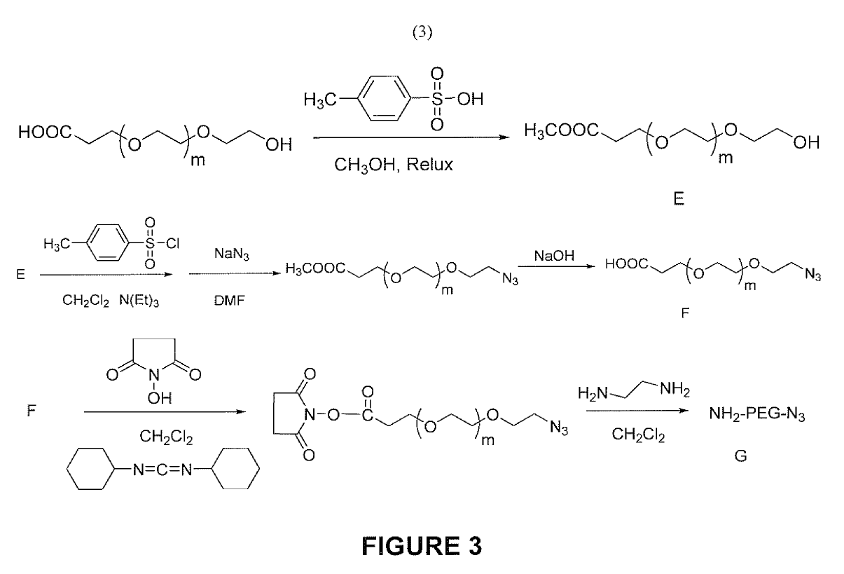

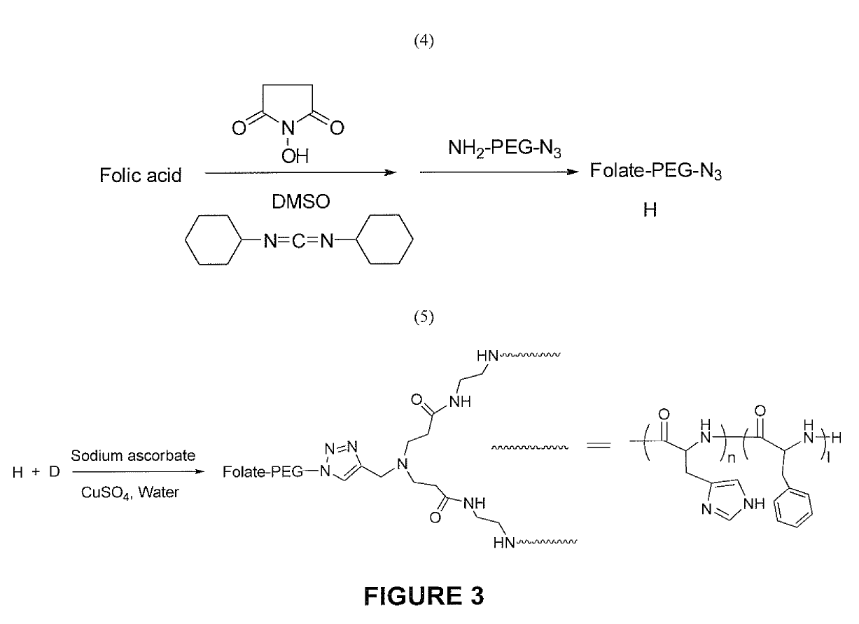

The targeting group can be covalently attached to the hydrophilic group of the polymer using techniques known in the art. For example, when the hydrophilic group is a PEG polymer, the polymer may include one or more reactive functional groups that are capable of reacting with targeting compound or drug molecule so that these molecules are covalently attached to the PEG polymer. Examples of such functional groups include hydroxyl, protected hydroxyl, active ester (e.g. N-hydroxysuccinimidyl, 1-benzotriazolyl, p-nitrophenyl, or imidazolyl esters), active carbonate (e.g. N-hydroxysuccinimidyl, 1-benzotriazolyl, p-nitrophenyl, or imidazolyl carbonate), acetal, aldehyde, aldehyde hydrates, alkyl or aryl sulfonate, halide, disulfide derivatives such as o-pyridyl disulfidyl, alkenyl, acrylate, methacrylate, acrylamide, active sulfone, amine, protected amine, hydrazide, protected hydrazide, thiol, protected thiol, carboxylic acid, protected carboxylic acid, isocyanate, isothiocyanate, maleimide, viniylsulfone, dithiopyridine, vinylpyridine, iodoacetamide, epoxide, glyoxals, diones, mesylates, tosylates, or tresylate. FIG. 3, equation 4 depicts one aspect of coupling a targeting group to the hydrophilic polymer. In this aspect, the targeting compound folic acid is coupled to H₂N-PEG-N₃ to produce compound H.

In other aspects, the hydrophobic group includes at least one pH-sensitive group bonded to it. The term “pH-sensitive group” is any group that can accept or donate a proton when the pH changes. For example, any group that is a Bronsted acid or base can be used as a pH-sensitive group herein. Examples of pH sensitive groups include, but are not limited to, an amino group, a hydroxyl group, a carboxylic acid group, or a heteroaryl group. Not wishing to be bound by theory, when the polymersomes possess one or more pH-sensitive groups, the bioactive agent incorporated in the polymersome can be released by changes in pH within the subject. The pH sensitivity of the polymersome can be modified by adjusting the number of pH-sensitive groups present in each polymer used to produce the polymersome. For example, decreasing the number of protonatable amino groups can reduce the amphiphilicity of the polymersome at neutral pH. In one aspect, the polymer used to produce the polymersome can have 1 to 400 protonatable amino or substituted amino or aromatic amino groups with molecular weights up to 50,000 Da. For example, the amino and/or substituted and/or imidazolyl amino groups (e.g., histidine) can be present in the hydrophobic portion of the polymer. In other aspects, the polymers used to produce the polymersome include 2, 3, 4, 5, 6, 7, 8, 9, or 10 histidine residues. Thus, the pH-sensitive amphiphilicity of the polymers and polymersomes produced by the polymers can be used to fine-tune the overall pKa of the polymersome. For example, low amphiphilicity of the polymersomes at physiological pH can minimize non-specific cell membrane disruption and nonspecific tissue uptake of the polymersomes containing the bioactive agent.

In one aspect, the polymer used to produce the polymersome has the formula II

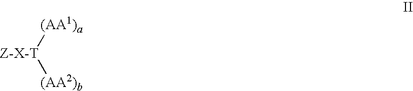

wherein (AA¹)ₐ and (AA²)_{b} are the same or different amino acid sequences;
a and b are each an integer from 5 to 500;
X includes a residue of a hydrophilic group;
T includes a residue derived from a group having at least three functional groups capable of forming a covalent bond with AA¹, AA² and X; and
Z includes a targeting agent.

With respect to the amino acids in formula II, in general the individual amino acids are linked to one another via amide bonds (—NC(O)—). The amino acid sequences AA¹ and AA² are the hydrophobic group as described above. The amino acids AA¹ and AA² can be composed of the same or different amino acid sequence. In certain aspects, the amino group of each amino acid is, independently, unsubstituted or substituted with an alkyl group, an alkenyl group, an acyl group, or an aromatic group.

The number of amino acids can vary depending upon the mechanism of delivering nucleic acids to cells. In one aspect, a and b in formula II are an integer from 5 to 500, 5 to 250, or 10 to 100. Any natural or non-natural amino acid can be used herein. In certain aspects, the amino acid sequence can be modified to include one or pH sensitive groups as described above. Alternatively, the amino acid can include a pH-sensitive group without chemical modification. In one aspect, at least one of AA¹ and AA² is histidine. In this aspect, the imidazole group present in histidine is the pH-sensitive group.

With respect to T in formula II, it is a residue derived from a group having at least three functional groups that are capable of forming covalent bonds. The functional groups in T can be the same or different. Examples of such functional groups include, but are not limited to, an amino group, a hydroxyl group, a carboxylic group, a carbonyl group, a carbamate group, a ureyl group, a thiol group, an alkynyl group, or an amide group. The compound can be any naturally-occuring or synthetic compound. The functional groups are capable of forming covalent bonds with the hydrophilic group X and hydrophobic groups (AA¹)ₐ and (AA²)_{b}.

In one aspect, the polymer used to make the polymersome has the formula III

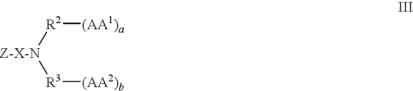

wherein AA¹ and AA² are the same amino acid sequence;
X is a residue of polyethylene glycol, and
R² and R³ are an alkyl amino group.

In this aspect, T as depicted in formula II is —N(R²)(R³), where R² and R³ are each an alkyl amino group. The term “alkylamino group” as used herein is any alkyl group as defined herein possessing at least one amino group, where the amino group can be substituted or unsubstituted. The alkyl groups can be branched or straight chain and can possess a plurality of amino groups. The amino groups of the alkyl amino group are capable of forming a covalent bond with an amino acid.

Examples of alkylamino groups useful herein include, but are not limited to, those depicted in formulae IV-VI

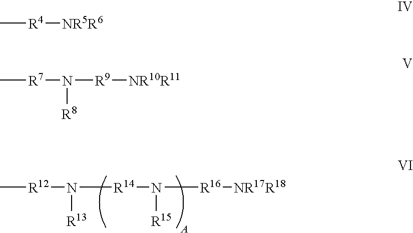

wherein R⁴, R⁷, R⁹, R¹², R¹⁴ and R¹⁶ are, independently, a straight chain or branched aliphatic hydrocarbon group, a cyclic aliphatic hydrocarbon group, or an aromatic group;
R^{5,} R⁶, R⁸, R¹⁰, R¹¹, R¹³, R¹⁵, R¹⁷ and R¹⁸ are, independently, hydrogen, an alkyl group, a nitrogen containing group, or a hydrophobic group; and
A is an integer from 1 to 50.

In another aspect, the alkylamino group can be: —CH₂NH₂, —CH₂CH₂NH₂, —CH₂CH₂CH₂NH₂, —(CH₂CH₂)₂NH, —(CH₂CH₂)₂NCH₃, —(CH₂CH₂)₂NCH₂CH₃, —(CH₂CH₂)₂NCH₂CH₂NH₂, —CH₂CH₂N(CH₂CH₂)₂NH, —CH₂CH₂N(CH₂CH₂)₂NCH₃, —CH₂CH₂N(CH₂CH₂)₂NCH₂CH₃, —CH₂CH₂CH₂CH₂NH₂, —CH₂CH₂CH₂CH₂CH₂NH₂, —CH₂NHCH₂CH₂CH₂NH₂, —CH₂CH₂NHCH₂CH₂CH₂NH₂, —CH₂CH₂CH₂NHCH₂CH₂CH₂CH₂NHCH₂CH₂CH₂NH₂, —CH₂CH₂NHCH₂CH₂CH₂CH₂NH₂, —CH₂CH₂NHCH₂CH₂CH₂NHCH₂CH₂CH₂NH₂, or —CH₂CH₂NH(CH₂CH₂NH)_{d}CH₂CH₂NH₂, where d is from 0 to 50

In another aspect, the trifunctional group T has the formula VII or VIII

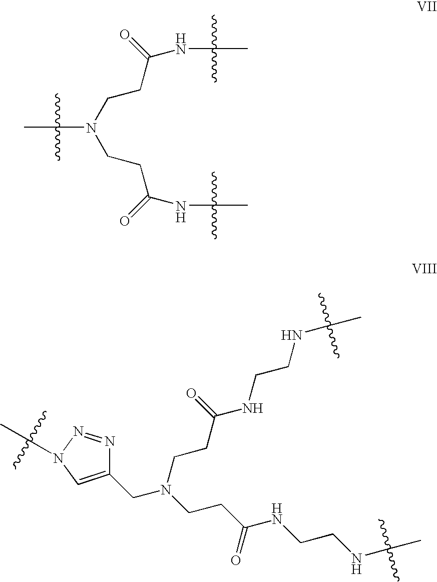

FIG. 3 provides an exemplary synthetic procedure for producing compounds having the formulae I and III, where (1) a targeting group is attached to the hydrophilic group PEG, and (2) pH-sensitive groups such as imidazole are present on the hydrophobic groups of the polymer. In equation 1 of FIG. 3, the trifunctional compound B is produced. In this example, the functional groups are two amino groups and an alkynyl group. In equation 2, protected forms of phenylalanine and histidine are coupled to the trifunctional compound B via the two amino groups to produce compound D. Equations 3 and 4 show techniques for attaching the targeting compound folic acid to PEG to produce compound H. Finally, equation 5 shows the coupling of compounds H and D to produce the final XY₂ polymer I with a targeting group attached to PEG and pH-sensitive groups (i.e., imidazole groups) attached to the hydrophobic groups.

Any of the polymers described herein useful in producing the polymersomes can be the pharmaceutically acceptable salt or ester thereof. Pharmaceutically acceptable salts are prepared by treating the free acid with an appropriate amount of a pharmaceutically acceptable base. Representative pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. The molar ratio of compounds of structural formula I to base used are chosen to provide the ratio desired for any particular salts. For preparing, for example, the ammonium salts of the free acid starting material, the starting material can be treated with approximately one equivalent of pharmaceutically acceptable base to yield a neutral salt.

Ester derivatives are typically prepared as precursors to the acid form of the compounds—as illustrated in the examples below—and accordingly can serve as prodrugs. Generally, these derivatives will be lower alkyl esters such as methyl, ethyl, and the like. Amide derivatives —(CO)NH₂, —(CO)NHR and —(CO)NR₂, where R is an alkyl group defined above, can be prepared by reaction of the carboxylic acid-containing compound with ammonia or a substituted amine.

A variety of techniques can be used to prepare the polymersomes described herein. The technique used varies depending upon the polymers and bioactive agents selected as well as the desired size of the polymersomes. In general, the bioactive agent is mixed with the polymers described herein prior to polymersome formation. The nature of the bioactive agent will determine where the bioactive agent is incorporated within the polymersome. If the bioactive agent is a hydrophobic compound, the compound will predominantly be present in the hydrophobic bilayer region of the polymersome. Conversely, if the bioactive agent is a salt (i.e., hydrophilic), it will likely be present in the aqueous core of the polymersome. In certain aspects, a hydrophilic bioactive agent can be incorporated within the core of the polymersome and a hydrophobic bioactive agent can be incorporated in the lipid bilayer of the same polymersome.

In one aspect, the thin film hydration method (TFH) can be used to make the polymersomes. In this aspect, a solution of polymer and bioactive agent in an organic solvent is prepared followed by evaporation of the solvent to produce a film. The film is next hydrated with an aqueous buffer solution followed by sonication to produce the polymersomes. Experimental procedures for making polymersomes by the TFH method are provided in the Examples.

In certain aspects, it is desirable to produce nano-sized polymersomes. For example, smaller polymersomes (20-200 nm or 50 nm to 200 nm in diameter) are desirable because these nano-vehicles are able to passively accumulate in solid tumors due to the enhanced permeability and retention effect. In one aspect, micro-sized polymersomes can be prepared initially and subsequently subjected to extensive sonication. Next, the polymersomes can be extruded multiple times through a membrane (e.g., polycarbonate membrane having a 100 nm pore size) to produce nano-sized polymersomes. Experimental procedures for making polymersomes by this methodology are provided in the Examples.

In another aspect, nano-precipitation can be used to produce the polymersomes. In one aspect, a solution of polymers and bioactive agent in an organic solvent are injected into an aqueous buffer solution by a micro-syringe under vigorous stirring to induce instantaneous microstructure formation, followed by through dialysis against aqueous buffer to remove the organic solvent. In this approach, nano-sized polymersomes can be directly produced. Experimental procedures for making polymersomes by this methodology are provided in the Examples.

A wide variety of bioactive agents can be incorporated in the polymersomes described herein and subsequently delivered to a subject. Examples of bioactive agents include, but are not limited to, nonsteroidal anti-inflammatory compounds, anesthetics, chemotherapeutic agents, immunotoxins, immunosuppressive agents, steroids, antibiotics, antivirals, antifungals, and steroidal antiinflammatories, anticoagulants. In one aspect, the bioactive agent can be a water-soluble therapeutic such as, for example, peptides, proteins, DNAs, siRNAs, miRNAs and Cisplatin). In another aspect, the bioactive agent can be water-insoluble therapeutic such as, for example, doxorubicin and pacalitaxel.

In one aspect, the bioactive agent is an anti-cancer drug. Anti-cancer drugs have numerous side-effects including nausea, neutropenia, and apolecia. Additionally, high doses of certain anti-cancer drugs (e.g., doxorubicin) can induce cardiac toxicity. The polymersomes described herein can provide a safe way to deliver to a subject an effective amount anti-cancer drug. Examples of anti-cancer drugs include, but are not limited to, platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine), antitumor antibiotics (e.g., daunorubicin, doxorubicin, idarubicin, epirubicin, mitoxantrone, bleomycin, mytomycin C, plicamycin, dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, sunitinib), monoclonal antibodies (e.g., rituximab, cetuximab, panetumumab, tositumomab, trastuzmab, alemtuzumab, gemtuzumab ozogamicin, bevacizumab), nitrosoureas (e.g., carmustine, fotemustine, and lomustine), enzymes (e.g., L-Asparaginase), biological agents (e.g., interferons and interleukins), hexamethylmelamine, mitotane, angiogenesis inhibitors (e.g., thalidomide, lenalidomide), steroids (e.g., prednisone, dexamethasone, and prednisolone), hormonal agents (e.g., tamoxifen, raloxifene, leuprolide, bicaluatmide, granisetron, flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, oxaprozin), selective cyclooxygenase-2 (COX-2) inhibitors, or any combination thereof.

In other aspects, the polymersomes described herein can be used to deliver nucleic acids to a subject. The nucleic acid can be an oligonucleotide, deoxyribonucleic acid (DNA), ribonucleic acid (RNA), or peptide nucleic acid (PNA). The nucleic acid of interest can be nucleic acid from any source, such as a nucleic acid obtained from cells in which it occurs in nature, recombinantly produced nucleic acid, or chemically synthesized nucleic acid. For example, the nucleic acid can be cDNA or genomic DNA or DNA synthesized to have the nucleotide sequence corresponding to that of naturally-occurring DNA. The nucleic acid can also be a mutated or altered form of nucleic acid (e.g., DNA that differs from a naturally occurring DNA by an alteration, deletion, substitution or addition of at least one nucleic acid residue) or nucleic acid that does not occur in nature.

In one aspect, the nucleic acid can be a functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, siRNA, miRNA, shRNA and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acids can be a small gene fragment that encodes dominant-acting synthetic genetic elements (SGEs), e.g., molecules that interfere with the function of genes from which they are derived (antagonists) or that are dominant constitutively active fragments (agonists) of such genes. SGEs can include, but are not limited to, polypeptides, inhibitory antisense RNA molecules, ribozymes, nucleic acid decoys, and small peptides. The small gene fragments and SGE libraries disclosed in U.S. Patent Publication No. 2003/0228601, which is incorporated by reference, can be used herein.

The functional nucleic acids can inhibit the function of an endogenous gene at the level of nucleic acids, e.g., by an antisense, RNAi or decoy mechanism. Alternatively, certain functional nucleic acids can function to potentiate (including mimicking) the function of an endogenous gene by encoding a polypeptide that retains at least a portion of the bioactivity of the corresponding endogenous gene, and may in particular instances be constitutively active.

Other therapeutically important nucleic acids include antisense polynucleotide sequences useful in eliminating or reducing the production of a gene product, as described by Tso, P. et al Annals New York Acad. Sci. 570:220-241 (1987). Also contemplated is the delivery of ribozymes. These antisense nucleic acids or ribozymes can be expressed (replicated) in the transfected cells. Therapeutic polynucleotides useful herein can also code for immunity-conferring polypeptides, which can act as endogenous immunogens to provoke a humoral or cellular response, or both. The polynucleotides employed according to the present invention can also code for an antibody. In this regard, the term “antibody” encompasses whole immunoglobulin of any class, chimeric antibodies and hybrid antibodies with dual or multiple antigen or epitope specificities, and fragments, such as F(ab)₂, Fab², Fab and the like, including hybrid fragments. Also included within the meaning of “antibody” are conjugates of such fragments, and so-called antigen binding proteins (single chain antibodies) as described, for example, in U.S. Pat. No. 4,704,692, the contents of which are hereby incorporated by reference.

In one aspect, the nucleic acid is siRNA. siRNAs are double stranded RNA molecules (dsRNAs) with approximately 20 to 25 nucleotides, which are generated by the cytoplasmic cleavage of long RNA with the RNase III enzyme Dicer. siRNAs specifically incorporate into the RNA-induced silencing complex (RISC) and then guide the RNAi machinery to destroy the target mRNA containing the complementary sequences. Since RNAi is based on nucleotide base-pairing interactions, it can be tailored to target any gene of interest, rendering siRNA an ideal tool for treating diseases with gene silencing. Gene silencing with siRNAs has a great potential for the treatment of human diseases as a new therapeutic modality. Numerous siRNAs have been designed and reported for various therapeutic purposes and some of the siRNAs have demonstrated specific and effective silencing of genes related to human diseases. Therapeutic applications of siRNAs include, but are not limited to, inhibition of viral gene expression and replication in antiviral therapy, anti-angiogenic therapy of ocular diseases, treatment of autoimmune diseases and neurological disorders, and anticancer therapy. Therapeutic gene silencing has been demonstrated in mammals, which bodes well for the clinical application of siRNA. It is believed that siRNA can target every gene in human genome and has unlimited potential to treat human disease with RNAi.

The polymersomes described above can be administered to a subject using techniques known in the art. For example, pharmaceutical compositions can be prepared with the polymersomes. It will be appreciated that the actual preferred amounts of the polymersome and bioactive agent incorporated therein in a specified case will vary according to the specific compound being utilized, the particular compositions formulated, the mode of application, and the particular situs and subject being treated. Dosages for a given host can be determined using conventional considerations, e.g., by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

Pharmaceutical compositions described herein can be formulated in any excipient the biological system or entity can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol.

Pharmaceutical carriers are known to those skilled in the art. These most typically would be standard carriers for administration to humans, including solutions such as sterile water, saline, and buffered solutions at physiological pH.

Molecules intended for pharmaceutical delivery can be formulated in a pharmaceutical composition. Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically, including ophthalmically, vaginally, rectally, intranasally. Administration can also be intravenously or intraperitoneally.

Preparations for administration include sterile aqueous or non-aqueous solutions, suspensions, and emulsions. Examples of non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Dosing is dependent on severity and responsiveness of the condition to be treated, but will normally be one or more doses per day, with course of treatment lasting from several days to several months or until one of ordinary skill in the art determines the delivery should cease. Persons of ordinary skill can easily determine optimum dosages, dosing methodologies and repetition rates.

It is understood that any given particular aspect of the disclosed compositions and methods can be easily compared to the specific examples and embodiments disclosed herein, including the non-polysaccharide based reagents discussed in the Examples. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

### Examples

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials

L-lactide, poly(ethylene oxide) methyl ether (PEO, Mₙ˜2000, 5000 Da), potassium tert-butoxide (^{t}BuOK), ethyl bromoacetate (BrCH₂CO₂Et), DEAE-Sephadex A-25, N-hydroxysuccinimide (NHS), N,N′-dicyclolhexylcarbodiimide (DCC), serinol, stannous octoate (SnOct₂), anhydrous toluene and dimethyl sulfoxide (DMSO) were purchased from Sigma-Aldrich Co. and used as received. FITC-Dextran (Mₙ˜4,000 Da) and Nile red were bought from Sigma and Fluka respectively and used without further purification. All the other chemicals were at least A.C.S. grade.

### Methods

Preparation of carboxyl-terminated PEO (FIG. 2, equation a). 10 mmol poly(ethylene oxide) methyl ether (PEO 2K, 5K Da) was dissolved in 100 ml toluene at 60° C., and 40 mmol BuOK was added. After 6 hr of reaction, 60 mmol BrCH₂CO₂Et was subsequently added. The reaction mixture was vigorously stirred at room temperature for 24 hr and was filtered; the filtrate was precipitated in cold diethyl ether and dried in vacuo. The obtained product was dissolved in 40 ml 1 M NaOH with 10 g NaCl added and the solution was stirred at room temperature for 2 hr, then acidified to pH 3 by addition of 6 M HCl. The solution was extracted with 40 ml dichloromethane, dried with anhydrous MgSO₄, and then precipitated in diethyl ether. Unreacted mPEG was finally removed by ion-exchange fractionation using a column containing DEAE-Sephadex A-25 tetraborate form to give pure carboxyl-terminated PEO. ¹H NMR (400 MHz, CDCl₃): δ (ppm)=3.38 (CH₃—O—CH₂—), 3.65 (—O—CH₂—CH₂—O—), 4.14(—O—CH₂—COOH).

Preparation of hydroxyl-terminated dendron (FIG. 2, equation b). 6 mmol NHS was added into the solution of 4 mmol carboxyl-terminated PEO in 20 mL dichloromethane under vigorous stirring, and 5 mmol DCC was subsequently added as an activator. The reaction mixture was stirred for 24 hr at room temperature while DCC was converted to N,N′-dicyclohexylurea which was filtered by precipitation at 4° C. The filtrate was precipitated in cold diethyl ether and dried in vacuo to give PEO-NHS. ¹H NMR (CDCl₃) δ (ppm)=2.85 (—N—CO—CH₂—CH₂—CO—), 3.38 (CH₃—O—CH₂—), 3.65 (—O—CH₂—CH₂—O—), 4.52 (—O—CH₂—CO—N—). Hydroxyl-terminated dendron was then obtained by coupling 1 mol activated PEO-NHS with excess serinol (7 mmol) in 20 ml DMSO. The reaction mixture was thoroughly dialysed against Mili-Q water using a semi-permeable membrane (Spectra/Por, MWCO: 2,000 Da) to remove unreacted serinol, subsequently extracted with 40 ml dichloromethane and precipitated in cold diethyl ether. Two times recrystallization of this precipitate eventually gave purified hydroxyl-terminated dendron (D₁). ¹H NMR (CDCl₃) δ (ppm)=3.38 (CH₃—O—CH₂—), 3.45-3.48 (CH₃—O—CH₂—CH₂—), 3.65 (—O—CH₂—CH₂—O—), 3.76-3.83 (—NH—CH—(CH₂—OH)₂), 4.03 (—O—CH₂—CO—NH—), 7.58 (—CO—NH—CH—).

Preparation of PEO-based AB₂ type miktoarm polymers (FIG. 2, equation c). The poly (L-lactic acid) terminated 3-miktoarm polymers PEO-b-(PLLA)₂ were synthesized from controlled ring-opening polymerization of L-lactide monomer using the above hydroxyl-terminated dendron (D₁) as initiators and SnOct₂ as catalyst. A typical example followed: 500 mg D₁ was first dissolved in 50 mL of toluene in a round-bottomed flask equipped with a Dean-Stark trap, followed by 1 hr of azeotropic distillation at 120° C. under a nitrogen atmosphere. After the solution was cooled down to 70° C., 500 mg L-lactide and SnOct₂ (1 wt. % of L-lactide) were added and the polymerization was carried out at 110° C. under a nitrogen atmosphere for 24 hr. The reaction was stopped by pouring in cold diethyl ether and the precipitate was purified by two times recrystallization from dichloromethane-diethyl ether. ¹H NMR (CDCl₃) δ (ppm)=1.58 (—CO—CH(CH₃)—O—), 3.38 (CH₃—O—CH₂—), 3.45-3.48 (CH₃—O—CH₂—CH₂—), 3.65 (—O—CH₂—CH₂—O—), 3.76-3.83 (—NH—CH—(CH₂—OH)₂), 4.03 (—O—CH₂—CO—NH—), 5.14-5.23 (—CO—CH(CH₃)—O—), 7.05 (—CO—NH—CH—). The degree of polymerization (DP) was estimated using integration ratio of the peaks from the repeating units —CO—CH(CH₃)—O— and —O—CH₂—CH₂—O— from the PLLA and PEO arms respectively. Detailed NMR and GPC characterizations of the 3-miktoarm polymers are shown in Table 1.

TABLE 1
Molecular and microstructural characterizations of the 3-miktoarm
PEG-b-(PLLA)₂ and diblock PEG-b-PLLA polymers.

| | M_{n(NMR)} | M_{n(GPC)} | | | M_{w}/ |
|---|---|---|---|---|---|
| Sample ID | (×10³ Da) | (×10³ Da) | DP_{EG}^{a} | DP_{LLA}^{a} | Mₙ^{a} |
| PEG-b-(PLLA)₂ | | | | | |
| PEG₄₅-b-(PLLA_{60.5})₂ | 11.3 | 10.7 | 45 | 121 | 1.3 |
| PEG₄₅-b-(PLLA_{32.5})₂ | 6.39 | 6.71 | 45 | 65 | 1.3 |
| PEG₁₁₇-b-(PLLA₃₈)₂ | 10.0 | 10.6 | 117 | 76 | 1.2 |
| PEG₄₅-b-(PLLA_{12.5})₂ | 3.44 | 3.79 | 45 | 25 | 1.2 |
| PEG₁₁₇-b-(PLLA_{17.5})₂ | 7.52 | 7.63 | 117 | 35 | 1.2 |
| PEG-b-PLLA | | | | | |
| PEG₄₅-b-PLLA₁₁₅ | 10.4 | 10.3 | 45 | 115 | 1.2 |
| PEG₄₅-b-PLLA₅₃ | 5.82 | 5.78 | 45 | 53 | 1.3 |
| PEG₄₅-b-PLLA₂₃ | 3.55 | 3.68 | 45 | 23 | 1.1 |
| PEG₁₁₇-b-PLLA₃₄ | 7.37 | 7.56 | 117 | 34 | 1.1 |

^{a}The degrees of polymerization of ethylene oxide (DP_{EG}) and L-lactic acid (DP_{LLA}), and polydispersity (M_{w}/Mₙ) were determined by GPC.

Preparation of PEO-b-PLLA diblock polymers. Diblock polymers were synthesized from controlled ring-opening polymerization of L-lactide monomer using poly(ethylene glycol) methyl ether (Mₙ: 2K, 5K) as initiator and SnOct₂ as catalyst, which has been reported in details elsewhere.^{[1]} ¹H NMR (CDCl₃) δ (ppm)=1.58 (—CO—CH(CH₃)—O—), 3.38 (CH₃—O—CH₂—), 3.45-3.48 (CH₃—O—CH₂—CH₂—), 3.65 (—O—CH₂CH₂—O—), 5.14-5.23 (—CO—CH(CH₃)—O—). The degree of polymerization (DP) was estimated using integration ratio of the peaks from the repeating units —CO—CH(CH₃)—O— and —O—CH₂—CH₂—O— from the PLLA and PEO arms respectively.

Dynamic light scattering (DLS). The sample was filtered prior to the measurement using a 0.80-μm disposable membrane filter. In DLS measurements, the measured unnormalized second-order autocorrelation function, G⁽²⁾(τ) can be expressed as the Siegert equation:

G⁽²⁾(τ)=A(1+b|g⁽¹⁾(τ)|²) (1)

where τ, A, b, and |g⁽¹⁾(τ)| are respectively the delay time, baseline, a coherence factor, and the normalized electric field time correlation function. The field correlation function was analyzed by the constrained regularized CONTIN method, to yield information on the characteristic linewidth (Γ) distribution G(Γ) from

|g⁽¹⁾(τ)|=∫G(Γ)e^{-Γτ}dΓ (2)

The first and second moments of G(Γ) are <Γ>=∫₀^{∞}ΓG(Γ)dΓ and μ₂=∫₀^{∞}(Γ−<Γ>)²G(Γ)dΓ, respectively. The value of μ₂/<Γ>² is a measure of the polydispersity, referred as polydispersity index (PDI). If the relaxation is diffusive, Γ can be related to the apparent translational diffusion coefficient Dₐₚₚ as

Dₐₚₚ=<Γ>/q² (3)

where q=4πn sin(θ/2)/λ is the magnitude of the scattering wave vector; n the solvent refractive index; λ the wavelength of the incident beam in vacuo (632.8 nm). The Dₐₚₚ is further correlated to the intrinsic diffusion coefficient D₀ via the following equation:

Dₐₚₚ=D₀(1+k_{d}c)(1+CR_{g}²q²) (4)

where k_{d} is the effective interaction parameter; c the polymer concentration; R_{g} the radius of gyration; C a parameter that is characteristic of the molecular architecture. Since k_{d}c can be negligible for a very dilute solution (less than 0.05 wt. % for all the samples), the intrinsic diffusion coefficient D₀ was obtained by extrapolation of experimental data of angular dependent diffusion coefficient (Dₐₚₚ) to zero angle

The hydrodynamic radius Rₕ is related to D₀ via the Stocks-Einstein equation:

D₀=kT/6πηRₕ (5)

where k is the Boltzmann constant and η is the viscosity of water at temperature T.

Static light scattering (SLS). SLS measurements were carried out at angles from 30 to 120°. The z-average gyration of radius <R_{g}> can be determined via the Rayleigh-Gans-Debye equation:

$$\frac{KC}{Δ R ( q )} \approx \frac{1}{{M}_{W}} ( 1 + \frac{< {R}_{g}^{2} >}{3} {q}^{2} ) ( 1 + 2 {A}_{2} {CM}_{W} ) \qquad ( 6 )$$

where K=4π²η²(dn/dC)²/(N_{A}λ⁴), C is the polymer concentration, dn/dC is the specific refractive index increment, N_{A} is Avogadro's number, ΔR(q) is the excess Rayleigh ratio, M_{w} is the weight-average molar mass, and A₂ is the second virial coefficient. ΔR(q) can be expressed as:

$$Δ R ( q ) = {{R}_{tol , 90} ( \frac{n}{{n}_{tol}} )}^{2} \frac{{I}_{ex} ( θ )}{{I}_{tol}} \qquad ( 7 )$$

where R_{tol,90} is the Rayleigh ratio of toluene at a measuring angle of 90°, Iₑₓ(θ)=(I−I₀)sin(θ) is the excess scattered intensity of the sample at the angle θ. Therefore a simplified Rayleigh-Gans-Debye equation is obtained as following:

$$\frac{1}{{I}_{ex}} \approx k ( 1 + \frac{< {R}_{g}^{2} >}{3} {q}^{2} ) \qquad ( 8 )$$

where k is an experimental constant.

Confocal laser scanning microscopy (CLSM). To prepare FITC-Dextran and Nile red loaded polymersomes by TFH method, a 1 mg.mL⁻¹ FITC-Dextran aqueous solution with 10 mM phosphate buffer saline was applied to hydrate the polymer film, followed by a 12 hr of dialysis using a dialysis membrane (MWCO: 10,000 Da) to remove free FITC-Dextran molecules. Subsequently, a 2 μL of Nile red solution in DMF (1 mg.mL⁻¹) was added to 2 mL of the FITC-Dextran loaded solution. After 1 day of equilibration one drop of the suspension was placed on an adhesive microscope slide and was viewed with an inverted CLSM equipped with an argon laser and a helium/neon mixed gas laser with excitation wavelengths of 488 and 543 nm, respectively. Clear differentiation between the fluorescence of the FITC labels and Nile red was achieved by optical band-pass filters of 505-530 nm and 605-660 nm for use in the channels of 488 and 543 nm respectively. Scans at a resolution of 1024×1024 pixels were taken in the XY mode.

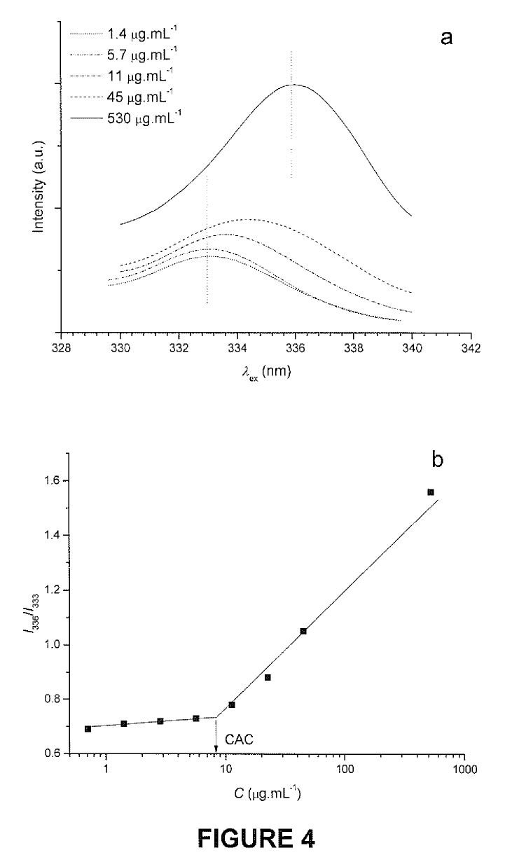

CAC measurements. Incorporation of pyrene into the polymeric micelles was achieved by injecting 2 μl pyrene in ethanol stock solution into 3 ml of micelle solution respectively, and the solution was stirred overnight. The final concentration of pyrene in the sample was 0.6 μM. The pyrene excitation at λ_{cm}=393 nm was recorded for CMC determinations. The fluorescence intensity ratios of two different bands (I₃₃₆/I₃₃₃) of pyrene are measured as a function of copolymer concentration. An increase in this ratio is observed at a given copolymer concentration, indicating the onset of polymer aggregation and thus the CAC. Representative plots of pyrene excitation spectra and variations of I₃₃₆/I₃₃₃ as a function of the copolymer concentration for PEG₄₅-b-(PLLA_{12.5})₂ are shown in FIG. 4.

Transmission electron microscopy (TEM). To prepare the samples, one drop of the aqueous solution was deposited onto a Formvar-carbon coated copper grid. 5 min later, excess solution was sucked away by touching the edge of the grid with a filter paper. The sample was then directly observed by TEM without staining.

Cryogenic transmission electron microscopy (cryo-TEM). In order to determine the nanoscale structure of the polymer sample, a high resolution TEM imaging analysis was performed by using a 300 kV JEM-3011 (Jeol, Tokyo, Japan) microscope equipped with a liquid nitrogen cryo-transfer holder system, enabling TEM observations at a specimen temperature of −140° C., and a low-scan CCD camera (Gatan, Pleasanton, Calif., USA). TEM samples were prepared by evaporation on the 200 mesh carbon/copper grid (Electron Microscopy Sciences, Hatfield, Pa., USA) by using a freeze dryer (Ilshinlab, Yangju, Korea) under a high-vacuum. After closure of the flask containing sample grid and temperature decrease, aqueous liquid and gases were trapped inside. Therefore, hydrated samples produced in a vacuum contained water and gases encapsulated under pressure. The freeze-dried samples were transferred into the cryotransfer holder with liquid nitrogen and this liquid inclusion was studied by using the TEM. The specific nanostructures apparent condensation and evaporation of hydrated samples were observed in situ.

Preparation of drug-loaded polymersomes. An aqueous solution of ammonium sulfate (155 mM, pH 7.4) was used to prepare nano-sized polymersomes by the sonication-extrusion method. The resulting samples were dialyzed in iso-osmotic Dulbecco's phosphate buffered saline (DPBS, modified, without calcium chloride and magnesium chloride, pH=7.4). Dialysis solutions were changed 3 times over approximately 12 hr. The polymersomes were then incubated with doxorubicin.HCl at a ratio of 1:0.2 polymer:drug (w/w) for 24 hours at room temperature. Non-entrapped DOX was removed by dialysis against DPBS buffer for another 12 hr.

### Results

Since the investigated PEG-b-(PLLA)₂ polymers do not readily dissolve in water, a conventional thin-film hydration (TFH) method was utilized to fabricate self-assemblies in aqueous solutions. A typical preparation procedure followed: 5 mg polymer was dissolved in 2 ml chloroform in a 10 ml round-bottom flask, which was then connected to a rotary evaporator to remove the solvent under a reduced pressure at 60° C. and kept under vacuum overnight at room temperature; 5 ml phosphate buffer (10 mM, pH 7.4) was subsequently added to the flask to hydrate the formed polymer film and the solution was stirred at 60° C. for 30 min, followed by another 10 min of sonication with a bath sonicator.

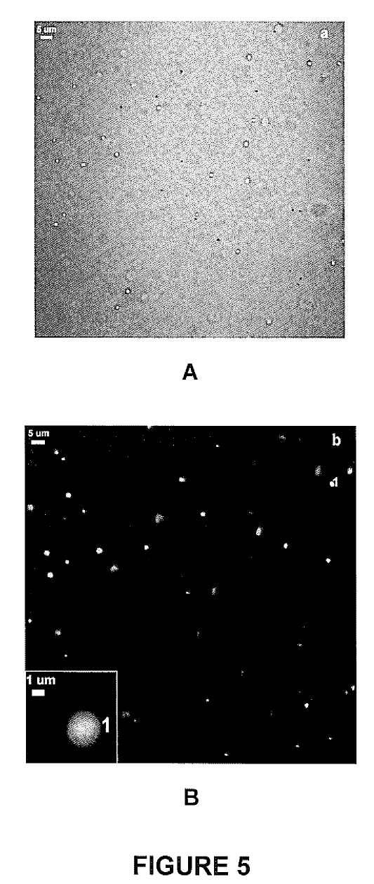

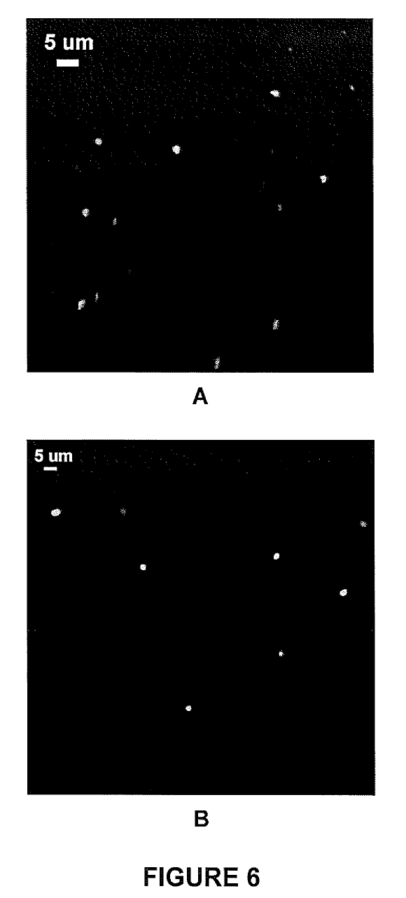

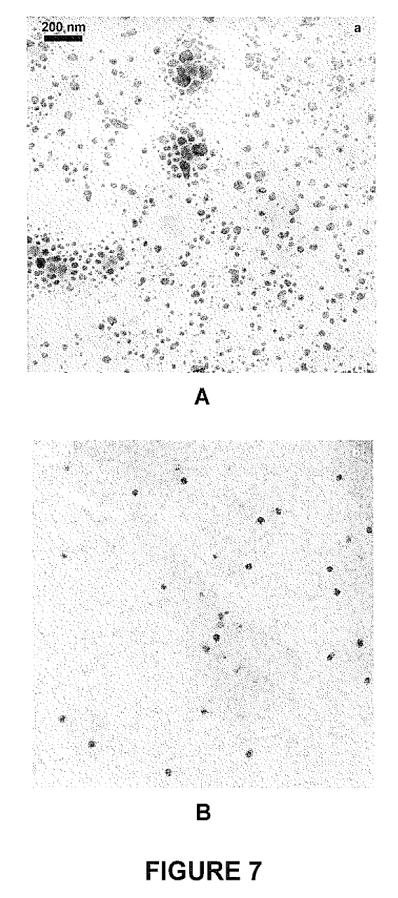

Laser scanning confocal microscopy (LSCM) was applied to study the formed microstructures. As shown in a representative differential interference contrast (DIC) micrograph (FIG. 5A), spherical particles with a diameter of 2-5 μm were observed for PEG₄₅-b-(PLLA_{32.5})₂. To further confirm their morphology, a hydrophobic fluorescence dye, Nile red, and a hydrophilic fluorescence dye, FITC-Dextran (Mₙ˜4,000 Da), were encapsulated into the particles. Under confocal microscope, the hydrophobic membrane of the vesicle and its aqueous lumen were clearly distinguished by the distinct fluorescence of the dyes (FIG. 5B). Micro-sized vesicle formation was also detected for all of the other 3-miktoarm copolymers (f_{EG}=0.2˜0.7; FIG. 6). Conversely, PEG-b-PLLA polymers with relatively low f_{EG} values (f_{EG}=0.21, 0.37) formed vesicles by the TFH method whereas micelles were found for greater f_{EG} values (f_{EG}=0.57, 0.70; FIG. 7), which is consistent with the previous studies on the diblock copolymers. Table 2 summarizes the molecular and microstructural characterizations of the 3-miktoarm PEG-b-(PLLA)₂ and diblock PEG-b-PLLA polymers.

TABLE 2
Molecular and microstructural characterizations of the 3-miktoarm
PEG-b-(PLLA)₂ and diblock PEG-b-PLLA polymers.

| | Mₙ^{a} | | | |
|---|---|---|---|---|
| Sample ID^{a} | (×10³ Da) | M_{w}/Mₙ^{a} | f_{EG}^{b} | Micro-structure^{c} |
| PEG-b-(PLLA)₂ | | | | |
| PEG₄₅-b-(PLLA_{60.5})₂ | 10.7 | 1.3 | 0.20 | V |
| PEG₄₅-b-(PLLA_{32.5})₂ | 6.71 | 1.3 | 0.32 | V |
| PEG₁₁₇-b-(PLLA₃₈)₂ | 10.6 | 1.2 | 0.51 | V |
| PEG₄₅-b-(PLLA_{12.5})₂ | 3.79 | 1.2 | 0.55 | V |
| PEG₁₁₇-b-(PLLA_{17.5})₂ | 7.63 | 1.2 | 0.70 | V |
| PEG-b-PLLA | | | | |
| PEG₄₅-b-PLLA₁₁₅ | 10.3 | 1.2 | 0.21 | V |
| PEG₄₅-b-PLLA₅₃ | 5.78 | 1.3 | 0.37 | V |
| PEG₄₅-b-PLLA₂₃ | 3.68 | 1.1 | 0.57 | SM + CM |
| PEG₁₁₇-b-PLLA₃₄ | 7.56 | 1.1 | 0.70 | SM |

^{a}The degree of polymerization for each PEG/PLLA block (indicated as the subscript), the number average molecular weight (Mₙ) and polydispersity (M_{w}/Mₙ) were determined by GPC.
^{b}The volume fraction of PEG was calculated using Mₙ(GPC) and the densities of bulk polymers at room temperature: ρ(PEG) = 1.13 g/cm³, ρ(PLLA) = 1.25 g/cm³.
^{c}V: vesicle; SM: spherical micelle; CM: compound micelle.

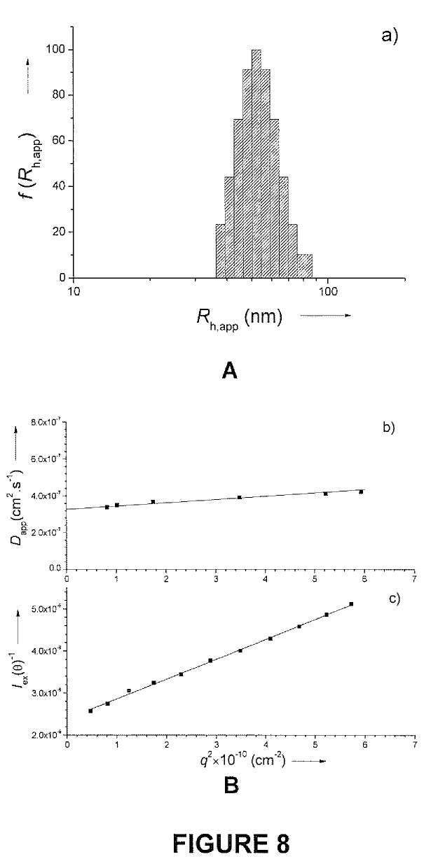

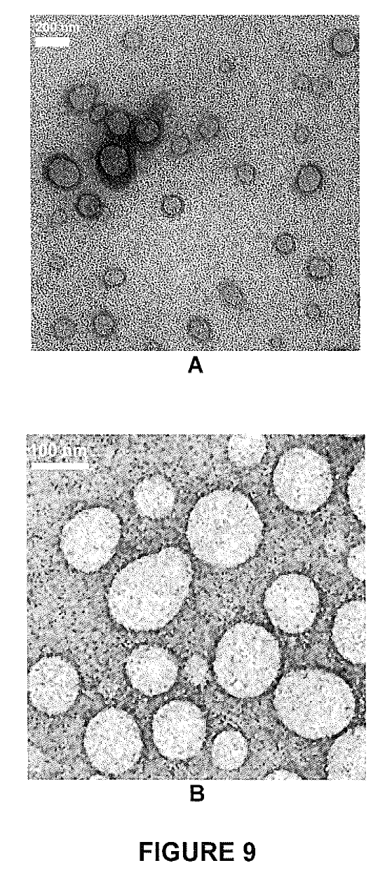

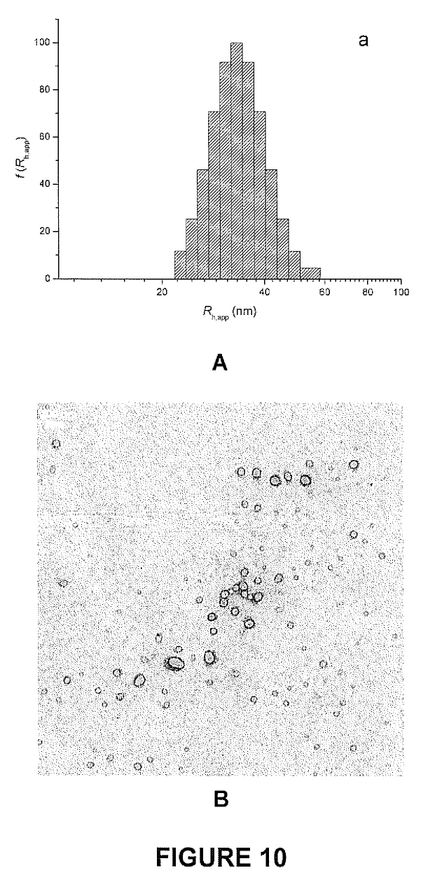

Nano-sized polymersomes from the PEG-b-(PLLA)₂ polymers were prepared by the following procedure. A “top-down” approach was first applied in which the prepared micro-sized polymersomes were subjected to extensive sonication up to 1 hr using a Microson probe sonicator (output power 2 W, 23 kHz), and were subsequently extruded multiple times through a polycarbonate membrane (100 nm pore size) with a Avanti mini-extruder. Consequently, nano-sized self-assemblies with a single diffusion mode and a relatively narrow size distribution (PDI=0.05˜0.1) were confirmed by dynamic light scattering (DLS) measurements (FIG. 8A). For PEG₄₅-b-(PLLA_{32.5})₂, a value of 73 nm of the hydrodynamic radius (Rₕ) was obtained by extrapolation of the apparent diffusion coefficient (Dₐₚₚ) to zero angle (FIG. 8B). Static light scattering (SLS) measurements were used to obtain the radius of gyration (R_{g}) based on the simplified Rayleigh-Gans-Debye equation and the R_{g} was determined as 74 nm (FIG. 8C). The value of the characteristic ratio R_{g}/Rₐ=1.04 close to 1 suggests the presence of hollow spheres. The microstructure of the nanoparticles was further studied by transmission electron microscopy (TEM). The micrographs of conventional TEM (FIG. 9A) and cryo-TEM (FIG. 9B) clearly revealed their spherical morphology and bilayer vesicular structure, and the size range was consistent with the light scattering measurements. More examples of nano-sized polymersomes formation with the other PEG-b-(PLLA)₂ molecules formed by this method can be found in FIG. 10.

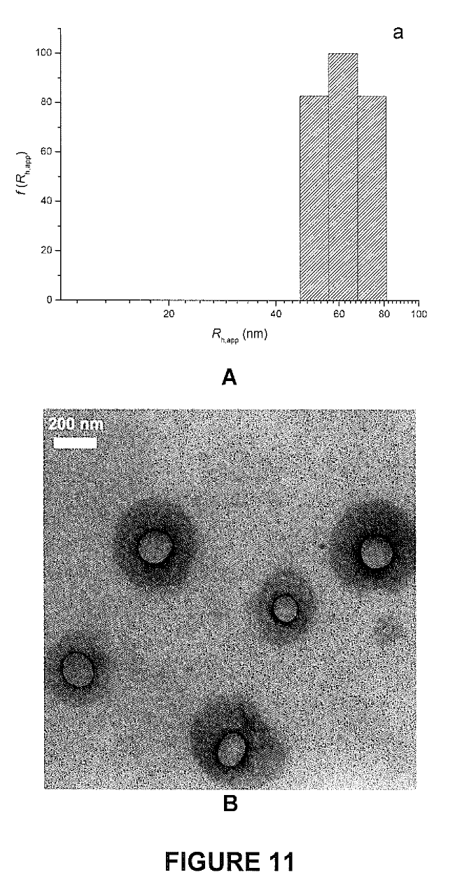

Alternatively, a nano-precipitation method was used to make nano-sized polymersomes. 50 μl of polymer solution in DMF (10 mg.mL⁻¹) was injected into 0.5 ml phosphate buffer by a micro-syringe under vigorous stirring to induce instantaneous microstructure formation, followed by through dialysis against aqueous buffer to remove the organic solvent. In this case, nano-sized polymersomes can be directly fabricated (FIG. 11).

Critical aggregation concentrations (CAC) for the PEG-b-(PLLA)₂ and PEG-b-PLLA polymers were measured respectively by a pyrene fluorescence probing method and the values are listed in Table 3.

TABLE 3
A comparison of CAC values between the PEG-b-(PLLA)₂ polymers
and their diblock (PEG-b-PLLA) counterparts.

| | CAC | | CAC |
|---|---|---|---|
| PEG-b-(PLLA)₂ | (μg · mL⁻¹) | PEG-b-PLLA | (μg · mL⁻¹) |
| PEG₄₅-b-(PLLA_{12.5})₂ | 8.3 ± 1.5 | PEG₄₅-b-PLLA₂₃ | 8.8 ± 0.8 |
| PEG₄₅-b-(PLLA_{32.5})₂ | 1.1 ± 0.2 | PEG₄₅-b-PLLA₅₃ | 1.3 ± 0.4 |
| PEG₄₅-b-(PLLA_{60.5})₂ | 0.55 ± 0.15 | PEG₄₅-b-PLLA₁₁₅ | 0.63 ± 0.11 |
| PEG₁₁₇-b-(PLLA_{17.5})₂ | 8.9 ± 2.3 | PEG₁₁₇-b-PLLA₃₄ | 9.1 ± 3.7 |

The PEG-b-(PLLA)₂ polymers were found to have small CMC values as low as several micrograms per milliliter, which will ensure good stability of the vesicles under in vitro/in vivo conditions. It is also worthy to note that the CAC value of a PEGₘ-(PLLAₙ)₂ polymer is close to that of its diblock counterpart i.e., PEGₘ-PLLA₂ₙ which has the same PEG block length and a similar PLLA composition. The dependence of CAC on molecular weight of PEG and PLLA also follows a similar trend for the two systems: an increase in the length of PLLA block leads to a significant decrease in the CAC value whereas a variation of the PEG length has little effect on that. The above results suggest that the superior vesicle-forming capability of the AB₂ 3-miktoarm block copolymer is probably closely related to its phospholipid-like structure.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed:

1. A polymersome comprising one or more bioactive agents, wherein the polymersome is derived from a polymer comprising the formula XY₂, wherein X comprises a hydrophilic group and Y comprises a hydrophobic group.

2. The polymersome of claim 1, wherein the hydrophilic group comprises a water soluble polymer block such as polyalkylene glycol, polyvinyl alcohol, polypyrrolidone, or a poly(amino acid).

3. The polymersome of claim 1, wherein the hydrophilic group comprises polyethylene glycol or a polyoxyethylene/polyoxypropylene copolymer.

4. The polymersome of claim 1, wherein the hydrophilic group comprises polyethylene glycol having a molecular weight from 500 to 20,000.

5. The polymersome of claim 1, wherein the hydrophilic croup comprises polyethylene glycol having a molecular weight from 2,000 to 10,000.

6. The polymersome of claim 1, wherein each hydrophobic group comprises, independently, a polyphosphazene, a polyphosphate ester, a polyanhydride, a polyhydroxybutyric acid, a polyorthoester, a poly(ε-caprolactone, a poly(α-hydroxy acid), or a copolymer prepared from two or more of these monomers.

7. The polymersome of claim 1, wherein each hydrophobic group comprises, independently, polylactic acid or poly(lactic-co-glycolic) acid having a molecular weight from to 500 to 10,000.

8. The polymersome of claim 1, wherein the polymer comprises the formula I 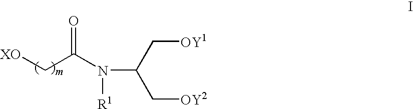
wherein X comprises a hydrophilic group;
Y¹ and Y² comprise hydrophobic groups;
m is from 1 to 5; and
R¹ is hydrogen, an alkyl group, or an aryl group.

9. The polymersome of claim 8, wherein the hydrophilic group is polyethylene glycol, Y¹ and Y² are, independently, polylactic acid, polyglycolic acid, poly(lactic acid-co-glycolic acid), poly(ε-caprolactone); m is one, and R¹ is hydrogen.

10. The polymersome of claim 1, wherein at least one targeting group is bonded to the hydrophilic group.

11. The polymersome of claim 10, wherein the targeting group comprises a protein, peptide, an antibody, an antibody fragment, one of their derivatives or other ligands that can specifically bind to receptors on targeted cells.

12. The polymersome of claim 1, wherein the hydrophobic group comprises at least one pH-sensitive group bonded to it.

13. The polymersome of claim 1, wherein the pH-sensitive group comprises an amino group, a hydroxyl group, a carboxylic acid group, or a heteroaryl group.

14. The polymersome of claim 1, wherein the pH-sensitive group comprises an imidazole group.

15. The polymersome of claim 1, wherein the polymer comprises the formula II 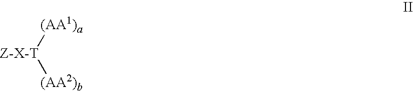
wherein (AA¹)ₐ and (AA²)_{b} are the same or different amino acid sequences;
a and b are each an integer from 5 to 500;
X comprises a residue of a hydrophilic group;
T comprises a residue derived from a group comprising at least three functional groups capable of forming a covalent bond with AA¹, AA² and X; and
Z comprises a targeting agent.

16. The polymersome of claim 15, wherein the polymer has the formula III 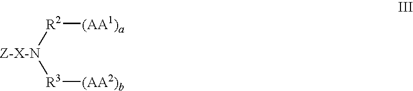
wherein AA¹ and AA² are the same amino acid sequence;
X is a residue of polyethylene glycol, and
R² and R³ are an alkyl amino group.

17. The polymersome of claim 16, wherein at least one of AA¹ and AA² is histidine.

18. The polymersome of claim 1, wherein the bioactive agent comprises an anti-cancer drug.

19. The polymersome of claim 1, wherein the polymersome has a diameter from 50 nm to 200 nm.

20. A pharmaceutical composition comprising the polymersome of claim 1.

21. A method for delivering a bioactive agent to a subject, the method comprising administering to the subject the polymersome of claim 1.

22. A polymer comprising the formula I 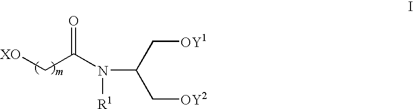
wherein X comprises a hydrophilic group;
Y¹ and Y² comprise hydrophobic groups;
M is from 1 to 5; and
R¹ is hydrogen, an alkyl group, or an aryl group.

23. A polymer comprising the formula II 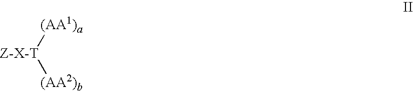
wherein (AA¹)ₐ and (AA²)_{b} are the same or different amino acid sequences;
a and b are each an integer from 5 to 500;
X comprises a residue of a hydrophilic group;
T comprises a residue derived from a group comprising at least three functional groups capable of forming a covalent bond with AA¹, AA² and X; and
Z comprises a targeting agent.
